(19) United States
(12) Patent Application Publication
(10) Pub. No.: US 2012/0283763 A1
(43) Pub. Date: Nov. 8, 2012

(54) Device with Echogenic Coating

(76) Inventors: Edward H. Cully, Flagstaff, AZ (US); Keith M. Flury, Flagstaff, AZ (US)

(21) Appl. No.: 13/465,354

(22) Filed: May 7, 2012

Related U.S. Application Data
(60) Provisional application No. 61/483,089, filed on May 6, 2011.

(51) Int. Cl. A61B 17/32; A61B 5/00

(52) U.S. Cl. 606/184; 427/2.12

(57) ABSTRACT

Devices with enhanced visualization in ultrasound imaging are provided.

### CROSS REFERENCE TO RELATED APPLICATIONS

This application claims priority to provisional application Ser. No. 61/483,089, filed May 6, 2011.

### FIELD OF THE INVENTION

The present invention relates to devices with enhanced echogenicity for better visualization in ultrasound imaging and methods for enhancing echogenicity of a device.

### BACKGROUND OF THE INVENTION

Ultrasound technology has advantages over other imaging modalities. Along with the health advantage of reducing or eliminating exposure to x-rays (fluoroscopy), the equipment needed is small enough to move and it has advantages in diagnosing sub-surface tissue morphology. Furthermore, ultrasound transducers can be made small enough to place, inside the body where they can provide better resolution than is currently available with magnetic resonance imaging and x-ray computed tomography. Further, interventional tool or device enhancements which increase their echogenicity to accommodate ultrasound enable clinicians to quickly and properly treat patients, saving time and money.

Many interventional tools and instruments are designed with polished surfaces that render the instruments virtually invisible on ultrasound. Interventional tools and instruments are herein referred to as “device(s)”. The present invention relates to an enhancement to increase echogenicity of interventional devices. Interventional devices include, but are not limited to, septal puncture needles as well as implantable devices, such as, but not limited to, stents, filters, stent graphs, and/or heart valves.

Ultrasound image device enhancement or “echogenicity” has been studied for many years. When sound waves contact a smooth surface, the angle of incidence and reflection are the same. If the object is located at a steep angle most or all the sound waves bounce away from a transmitting/receiver source. With such steep angles, even highly reflective devices can be invisible by ultrasound if scattering does not direct sound back to a source transducer. Conversely, if an object is perpendicular, the sound waves reflecting directly back may cause a “white out” effect and prevent the operator from seeing around the object. This affect is referred to as specular reflection.

Medical device manufacturers have tried a variety of techniques to improve visibility of devices to ultrasound. Examples include roughening the surface of the device, entrapping gas, adhering particles to substrate surfaces, creating indentations or holes in the substrates and using dissimilar materials.

### SUMMARY OF THE INVENTION

An aspect of the present invention relates to an echogenically enhanced interventional tool or device. The interventional tool or device to be imaged ultrasonically has an outer surface with a fused polymer particle coating affixed to at least a portion of the outer surface of the tool or device.

Another aspect of the present invention relates to a method for enhancing echogenicity of an interventional tool or device through the attachment of biological elements to the surface of the interventional tool or device. In this embodiment, the interventional tool or device may have an initially smooth surface onto which biological elements attach; thereby increasing the surface roughness and echogenicity.

Another aspect of the present invention relates to a method for enhancing echogenicity of an interventional tool or device. In this method, a fused polymer particle coating is affixed to at least a portion of the interventional tool or device.

### BRIEF DESCRIPTION OF THE FIGURES

FIG. 1 shows an interventional tool or device.

FIG. 2 shows the same interventional tool or device of FIG. 1 coated with fused polymer particles.

FIG. 3 is a bar graph showing results of a comparison of the dB increase above control of a device of the present invention with a fused polymer particle coating as depicted in FIG. 2, a spray coating of a solvated polymer, and another commercially available coated device.

FIG. 4 is a plot of the reflected energy at various angles, which reflects increased echogenic response.

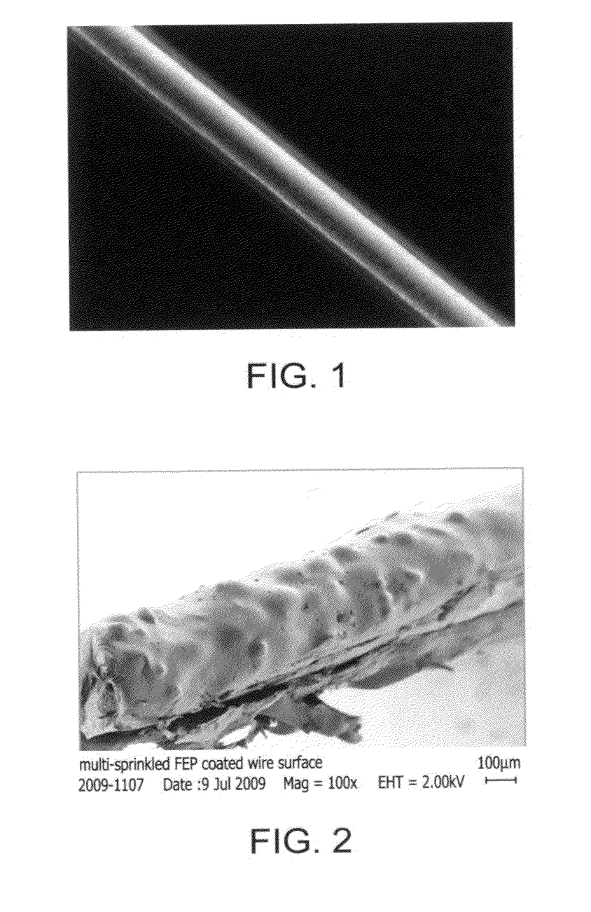

### DETAILED DESCRIPTION OF THE INVENTION

The echogenically enhanced device of the present invention comprises a device to be imaged ultrasonicallyhaving an outer surface at least a portion of which is affixed with a fused polymer particle coating.

Examples of interventional tools or devices which can be enhanced visually in ultrasound imaging in accordance with the present invention include, but are not limited to, medical devices such as permanent implantable or temporary indwelling devices, such as catheters, guide wires, stents and other accessories and tools, surgical instruments, and needles, such as septal puncture needles. However, as will be understood by the skilled artisan upon reading this disclosure, the techniques described herein for visually enhancing a device via ultrasound imaging are adaptable to many different fields and devices.

Echogenicity of this device is enhanced in accordance with the present invention by affixing to at least a portion of the outer surface of the device a fused polymer particle coating.

In one embodiment, the fused polymer particles of the coating are at least partially interconnected. Depending on the degree of echogenic enhancement desired, lower concentrations of polymer particles may be employed so that some particles while adhered to the device may not be fused to an adjacent polymer particle or particles.

In one embodiment, the fused polymer particle coating provides an irregular surface topography on the outer surface of the tool or device. This irregular surface topography produces a unique, visible signature on the device when viewed with ultrasound. Depending on the end device application, an alternate desirable embodiment may include areas of fused polymer particles wherein the topography is flat and/or even concave.

In one embodiment, the fused polymer particle coating has a surface roughness greater than 0.5% of a selected ultrasonic imaging wavelength. For example, for ultrasonic imaging at 7.5 MHz the wavelength is 200 μm. Thus at this ultrasonic wavelength, in this embodiment the fused polymer particle coating has a surface roughness of greater than 1 μm (0.5% of 200 μm).

One embodiment of the fused polymer particle coating may comprise fused fluoropolymer particles, fused silicone particles, fused polyolefin particles, and the like. Examples of fused fluoropolymer particles for use in the coatings of the present invention include, but are not limited to, fluorinated ethylene propylene (FEP) and fluorinated ethylene propylene perfluora alkyl vinyl ether, or polytetrafluoroethylene-co-vinyl acetate. Of particular interest is a particle of PTFE (which can be screened to an exact and certain size). The PTFE particles are then bonded to the medical device or implant with FEP or ethylene fluoroethylene propylene (EFEP). It is possible to achieve the same results by using differing melt flow indices of the same polymer.

In one embodiment, the fused polymer particle coating has a melt temperature of less than 300° C. In another embodiment, the fused polymer particle coating has a melt temperature of less than 200° C. In yet another embodiment, the fused polymer particle coating has a melt temperature of less than 170° C. In another embodiment, the fused polymer particle coating has a melt temperature of less than 140° C. In other embodiments, the fused polymer particle has an amorphous state with no defined melt temperature.

An important aspect of the present invention is the need to select polymer particles that wilt produce a fused particle coating without adversely affecting the nature and function of the device to be imaged.

In another embodiment of the present invention, the echogenicity of an interventional tool or device is enhanced through the attachment of biological elements to the surface of an interventional tool or device. In this embodiment, the interventional tool or device may have an initially smooth surface onto which biological elements, attach. Biological elements include blood cell, fibrin, platelets, and the like. To encourage the attachment of biological elements, the interventional tool or device may comprise a surface coating such as fibrin or positive charges by means such as, but not limited to a thin polyethylene imine coating.

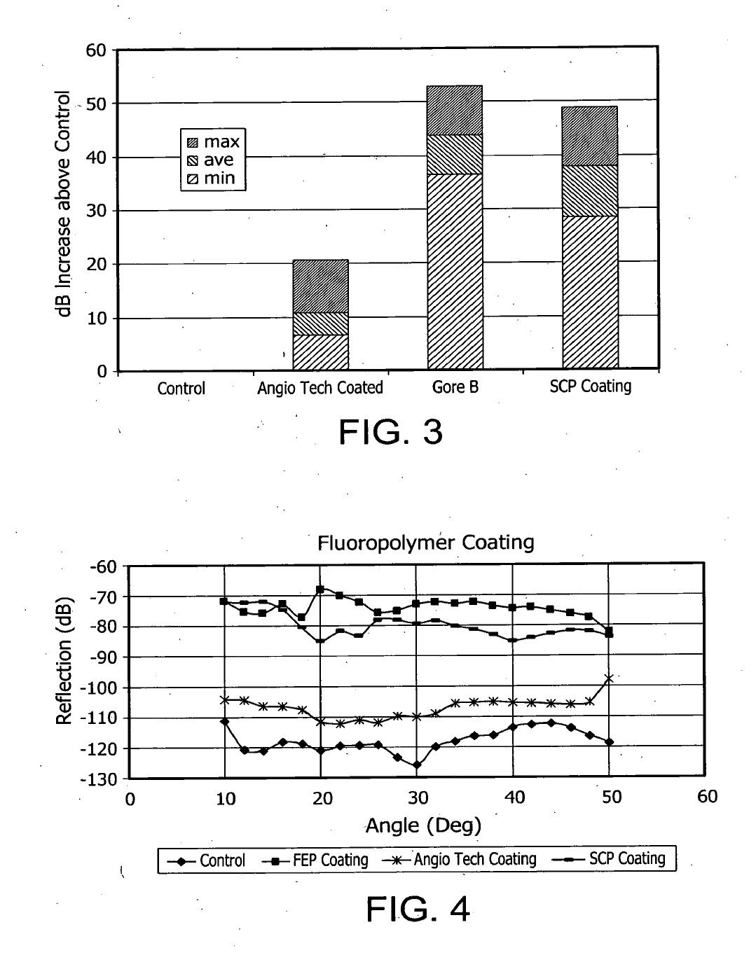

Enhanced echogenicity of a device according to an aspect of the present invention was demonstrated experimentally. Results are depicted in FIG. 3 which shows a comparison of the dB increase above control of a device according to an aspect of the present invention and an Angiotech coated device.

The following non-limiting examples are provided to further illustrate the present invention.

### EXAMPLES

### Example 1

### Materials

A stainless steel needle with the dimensions of 0.040″ diameter and approximately 4.8″ long was used as the test article for echogenic enhancement. An unmodified needle was used as control to compare the results of the modification. Echogenicity of a stainless steel needle coated with a fused polymer particle coating was also compared to an Angiotech coated needle (Angiotech Pharmaceuticals, Inc., 1618 Station Street, Vancouver, BC Canada V6A 1B6). In addition, a second embodiment was prepared by dissolving a thermoplastic copolymer of TFE and PMVE in solution as described in U.S. Pat. No. 7,049,380. This solution was sprayed at a rate of 2 ml/min. using a sprayer (Air Atom, Spray Systems Co.) set to 28.2 psig air pressure to form a fine mist. A smooth needle was then slowly rotated and passed back and forth through this spray mist for a total of three passes. The solvent was, air dried. The topography of this spray coated device was increased relative to the base, smooth device surface. The echogenic response of the coated needle is plotted in FIGS. 3 and 4, which reflect the increased echogenic response of the coated needle.

### Example 2

### Methods

Three different methods were used to evaluate and compare the treated samples.

All samples were subjected to an acoustic wave imaging system. The testing apparatus consisted of a 7.5 MHz transmitting/receiving transducer mounted onto a flat bar with a sample holder placed approximately 2.5 cm at the transducer's focal length. The 7.5 MHz transducer produced a wave length (λ) of 200 microns. At 2.5 cm the width of the signal was approximately 1 mm. The needle sample was placed into a holder that is perpendicular to the axis of the emitting transducer. This is 0 degrees. The sample holder is removable for ease of changing out the sample. The holder is magnetically held in a rotatable device for measuring the angle of the sample relative to the transmitting and receiving transducer. The sample and transducer were submerged into a room temperature water tank. Before collecting the data, every sample was aligned with the transducer. This was accomplished by increasing the attenuation setting on the pulser/receiver controller (approximately 40 dB) to prevent saturation of the received signal. The operator then visually monitored the wave signal while manually rotating the goniometer and dialing the fine adjustment knobs on the transducer to achieve a maximum return signal. The attenuation was adjusted to a reference point of approximately 1 volt. The attenuation setting and the goniometer indication were recorded. The goniometer was rotated 10 degrees from the recorded indication. Since the signal typically decreases off of perpendicular (specular reading) the attenuation was reduced. The reduced level allowed a strong enough signal during collection, without saturation of the receiver. The sample was rotated through the entire angular rotation to ensure that the signal did not saturate or significantly move away from or closer to, the transducer moving the signal out of the data collection window. Significant time shift was an indication that the transducer was not aligned with the center or pivot of the sample. Once the set-up was completed, the goniometer was moved to the 10 degree mark and the collection of points was taken to 50 degrees at 2 degree increments. Equipment connected to the transducer and test fixture measured reflection. The software, Lab View and hardware were used for data collection and later analysis.

A second evaluation of samples was performed in a silicone phantom submersible in a blood substitute from ATS laboratories to increase attenuation and create a more realistic image environment. Using a 6.5 mHz transducer ultrasound system, the samples were inserted into the phantom. A still image was captured for each sample. These images were visually compared to control images and inspected for consistency with the transducer 2D data. The data was collected at three different times. Between collections two and three, the transducer was rebuilt. Thus, while the absolute dB scale of plots is not the same, the relative deltas are of importance.

The third evaluation was a surface analysis using an optical comparator. All raw data was further processed by the machine software to better evaluate the samples. The macroscopic tilt and cylindrical curvature, were removed. A Gaussian filter (Fourier) was selected to filter frequencies below 20⁻¹ mm. Incomplete interior points were restored with a maximum of 3 or 5 pixels. All samples were masked at the edges to remove large data drop out sections and anomalies associated with the filtering. 2D samples were processed first followed by 3D samples.

Total roughness height, Rt or PV, which is the maximum peak to valley height of the surface profile within the assessment length, was used to characterize surface roughness.

A comparison of the dB increase above control of a device of the fused particle embodiment, the SCP coated embodiment, and an Angiotech coated device is depicted in FIG. 3.

What is claimed is:

1. An echogenically enhanced device comprising:
(a) an device having an outer surface; and
(b) a fused polymer particle coating affixed to at least a portion of the outer surface of said tool or device.

2. The echogenically enhanced device of claim 1 wherein the fused polymer particles of the coating are at least partially interconnected.

3. The echogenically enhanced device of claim 1 wherein the fused polymer particle coating provides an irregular surface topography on the outer surface of the tool or device.

4. The echogenically enhanced device of claim 1 wherein the fused polymer particle coating has a surface roughness greater than 0.5% of a selected ultrasonic imaging wavelength.

5. The echogenically enhanced device of claim 1 wherein the fused polymer particle coating has a surface roughness greater than 1.0 μm.

6. The echogenically enhanced device of claim 1 wherein the fused polymer particle coating comprises fused fluoropolymer particles.

7. The echogenically enhanced device of claim 6 wherein the fused fluoropolymer particles comprise fluorinated ethylene propylene or fluorinated ethylene propylene.

8. The echogenically enhanced device of claim 1 wherein the fused polymer particle coating has a melt temperature of less than 300° C.

9. The echogenically enhanced device of claim 1 wherein the fused polymer particle coating has a melt temperature of less than 200° C.

10. The echogenically enhanced device of claim 1 wherein the fused polymer particle coating has a melt temperature of less than 170° C.

11. The echogenically enhanced device of claim 1 wherein the device is a surgical instrument.

12. The echogenically enhanced device of claim 1 wherein the device is a septal puncture needle.

13. A method for enhancing echogenicity of an interventional tool or device, said method comprising:
affixing a polymer particle coating to at least a portion of a device.

14. The method of claim 13 wherein the fused polymer particle coating comprises fused fluoropolymer particles.
